(19) United States
(12) Patent Application Publication
(10) Pub. No.: US 2014/0074065 A1
(43) Pub. Date: Mar. 13, 2014

(54) Bioabsorbable Spacers and Spacer Delivery Systems for Use in the Ear, Nose and Throat

(71) Applicant: ACCLARENT, INC., Menlo Park, CA (US)

(72) Inventors: Ketan P. Muni, San Jose, CA (US); Sandra W. Ruggles, Sunnyvale, CA (US); Jeffrey S. Jones, San Mateo, CA (US); Hung V. Ha, San Jose, CA (US); Howard Levine, Longboat Key, FL (US); Joseph H. Contiliano, Stewartsville, NJ (US); Joshua Makower, Los Altos, CA (US); Shrirang V. Ranade, Foster City, CA (US)

(21) Appl. No.: 13/800,211

(22) Filed: Mar. 13, 2013

Related U.S. Application Data
(60) Provisional application No. 61/698,360, filed on Sep. 7, 2012.

(51) Int. Cl. A61F 2/82

(52) U.S. Cl. CPC A61F 2/82
USPC 604/514; 604/93.01; 606/199; 264/154

(57) ABSTRACT

A spacer for delivery into the natural or man-made openings to the frontal, maxillary, sphenoid, anterior or posterior ethmoid sinuses, or other cells or cavities, anatomical regions such as nostrils, nasal cavities, nasal meatus, and other passageways such as the Eustachian tubes, naso-lachrymal ducts or airway is described. The bioabsorbable polymeric spacers maintain the opening and/or are useful for delivering drugs or other substances to the natural or man-made openings.

### FIELD OF THE INVENTION

The present invention relates, in general, to medical devices and, in particular, to bioabsorbable spacers and methods and devices for delivering the spacers for the treatment of conditions of the ear, nose, and throat.

### BACKGROUND OF THE INVENTION

The paranasal sinuses are hollow cavities in the skull connected by small openings, known as ostia, to the nasal canal. Each ostium between a paranasal sinus and the nasal cavity is formed by a bone covered by a layer of mucosal tissue. Normally, air passes into and out of the paranasal sinuses through the ostia. Also, mucus is continually formed by the mucosal lining of the sinuses and drains through the ostia and into the nasal canal.

Sinusitis is a general term that refers to inflammation in one or more of the paranasal sinuses. Acute sinusitis can be associated with upper respiratory infections or allergic conditions, which may cause tissue swelling and temporarily impede normal trans-ostial drainage and ventilation of the sinuses, thereby resulting in some collection of mucus and possibly infection within the sinus cavities. Chronic sinusitis is a long term condition characterized by persistent narrowing or blockage of one or more sinus ostia, resulting in chronic infection and inflammation of the sinuses. Chronic sinusitis is often associated with longstanding respiratory allergies, nasal polyps, hypertrophic nasal turbinates and/or deviated nasal septum. While acute sinusitis is typically caused by infection with a single pathogen (e.g., one type of bacteria, one type of virus, one type of fungus, etc.), chronic sinusitis is often associated with multiple pathogen infections (e.g., more than one type of bacteria or more than one genus of micro-organism).

Chronic sinusitis, if left untreated, can result in irreparable damage to the tissues and/or bony structures of the paranasal anatomy. The initial treatment of chronic sinusitis usually involves the use of drugs such as decongestants, steroid nasal sprays and antibiotics (if the infection is bacterial). Various drugs have been used to treat sinusitis, including systemic antibiotics. Intranasal corticosteroid spray and intranasal decongestant spray and drops have also been used. However, the use of intranasal sprays and drops by most patients does not result in the drug actually entering the affected intranasal sinuses. Rather such sprays and drops typically contact only tissues located within the nasal cavity. The introduction of drugs directly into the sinuses has been proposed by others, but has not become a widely used treatment technique.

In cases where drug treatment alone fails to provide permanent relief, surgical intervention may be indicated. The most common surgical procedure for treating chronic sinusitis is functional endoscopic sinus surgery (FESS). FESS is commonly performed using an endoscope and various rigid instruments inserted through the patient's nostril. The endoscope is used to visualize the positioning and use of various rigid instruments used for removing tissue from the nasal cavity and sinus ostia in an attempt to improve sinus drainage.

Functional endoscopic sinus surgery (FESS) that my also include polypectomy (and associated tissue injury) will often cause polyps to rebound quickly, negating the positive effects of the surgery. Stasis in the post operative period can result in mucus plugs that require physical removal. The provision of a drainage pathway is critical to avoid crusting and adhesions. There is an unmet clinical need to have a better way to clear mucus following FESS and minimize post operative encrustation, obstruction and adhesion.

In addition, following FESS or balloon sinuplasty, the opening to the sinus can re-narrow with healing and scar formation. It is desirable to have a functional drainage pathway through the ostiomeatal complex to ensure long term function via maintenance of the patency of the drainage pathway.

A technique known as the Balloon Sinuplasty™ procedure and a system for performing the procedure has been developed by Acclarent Inc, of Menlo Park, Calif. for treatment of sinusitis. A number of US patents and patent applications including U.S. Pat. Nos. 7,645,272, 7,654,997, and 7803150 describe various embodiments of the Balloon Sinuplasty™ procedure as well as various devices useable in the performance of such procedure. In the Balloon Sinuplasty™ procedure, a guide catheter is inserted into the nose and positioned within or adjacent to the ostium of the affected paranasal sinus. A guidewire is then advanced through the guide catheter and into the affected paranasal sinus. Thereafter, a dilation catheter having an expandable dilator (e.g. an inflatable balloon) is advanced over the guidewire to a position where the dilator is positioned within the ostium of the affected paranasal sinus. The dilator is then expanded, causing dilation of the ostium and remodelling of bone adjacent to the ostium, without incision of the mucosa or removal of any bone. The catheters and guidewire are then removed and the dilated ostium allows for improved drainage from and ventilation of the affected paranasal sinus.

Following FESS or Balloon Sinuplasty, adhesions may form between mucosal surfaces that have been disturbed. Accordingly, it would be desirable to insert a spacer into the natural or man-made openings to the frontal, maxillary, sphenoid, anterior or posterior ethmoid sinuses, or other cells or cavities of the ear, nose or throat, anatomical regions such as nostrils, nasal cavities, nasal meatus, etc., and other passageways such as the Eustachian tubes, nasolacrimal ducts or airway to maintain the opening and/or for delivering drugs or other substances to the natural or man-made openings.

### SUMMARY OF THE INVENTION

In a first aspect, the invention is a spacer for maintaining the patency of an opening in the sinus anatomy. The spacer may be fabricated from a bioabsorbable material such as a co-polymer of epsilon-caprolactone and glycolide where the co-polymer has between 10% and 35% by weight of epsilon-caprolactone.

In one embodiment, the spacer is in the shape of a cylinder. In another embodiment, the cylinder has a proximal end and a distal end and the distal end comprises a flare having an angle of between 5 degrees and 180 degrees. In still another embodiment, the proximal end of the spacer further comprises a flare.

In a further embodiment, the cylinder comprises a scaffolding, and in another embodiment the scaffolding comprise members are selected from the group consisting of longitudinal members, axial members and a combination of longitudinal and axial members.

In another embodiment, the spacer has an initial lateral stiffness of between about 0.2 and 5 N/mm and in another embodiment the initial lateral stiffness is between about 0.5 and 2 N/mm.

In still another embodiment, the spacer includes a therapeutic or diagnostic substance and in another embodiment the therapeutic substance is triamcinolone acetonide.

In another aspect, the invention is a method of making a spacer for maintaining the patency of an opening in the sinus anatomy. The method includes melt extrusion of a copolymer of epsilon-caprolactone and glycolide to make a tubing of between 6 mm and 7 mm in diameter with a thickness of between 0.25 mm an 2.0 mm, micromachining the tubing to produce micro-machined tubing, cutting the micromachined tubing into lengths of between about 8 mm and 15 mm; and heat setting the micromachined tubing lengths to produce a flare with a flare cone angle of between 5° to 180°.

In still another aspect, the invention is a method of deploying a spacer into an opening in the sinus anatomy to maintain the patency of the opening. The method includes compressing a spacer having an outer diameter of between 6 mm and 7 mm into a delivery device having an inner diameter of between 2 mm and 3 mm diameter, inserting the delivery device into the sinus anatomy, and deploying the spacer such that the spacer expands into the sinus anatomy to an outer diameter of between 6 mm and 7 mm, sufficiently to maintain patency of the opening in the sinus anatomy.

In one embodiment, the spacer expansion may be accomplished by self-expansion, balloon expansion or a combination of self-expansion and balloon-expansion.

In still another aspect, the invention is a method for delivering triamcinolone acetonide to the frontal sinus anatomy. The method includes compressing a spacer having an outer diameter of between 6 mm and 7 mm and comprising triamcinolone acetonide into a delivery device having an inner diameter of between 2 mm and 3 mm diameter, inserting the delivery device into the frontal sinus anatomy, and deploying the spacer such that the spacer expands into the frontal sinus anatomy to an outer diameter of between 6 mm and 7 mm and delivers the triamcinolone acetonide into the frontal sinus anatomy.

In one embodiment, the spacer expansion may be accomplished by self-expansion, balloon expansion or a combination of self-expansion and balloon-expansion.

In another aspect, the invention is a spacer for maintaining an opening in the anatomy of a human patient that is accessible from the ear, nose or throat. The spacer may be fabricated from a bio-absorbable material such as a co-polymer of epsilon-caprolactone and glycolide where the co-polymer has between 10% and 35% by weight of epsilon-caprolactone.

In one embodiment, the spacer is in the shape of a cylinder. In another embodiment, the cylinder has a proximal end and a distal end and the distal end comprises a flare having an angle of between 5 degrees and 180 degrees. In still another embodiment, the proximal end of the spacer further comprises a flare.

In a further embodiment, the cylinder comprises a scaffolding, and in another embodiment the scaffolding comprise members are selected from the group consisting of longitudinal members, axial members and a combination of longitudinal and axial members.

In another embodiment, the spacer has an initial lateral stiffness of between about 0.2 and 5 N/mm and in another embodiment the initial lateral stiffness is between about 0.5 and 2 N/mm.

In still another embodiment, the spacer includes a therapeutic substance.

In another aspect, the invention is a method of making a spacer for maintaining an opening in the human anatomy. The method includes melt extrusion of a copolymer of epsilon-caprolactone and glycolide to make a tubing of between 0.5 mm and 25 mm in diameter with a thickness of between 0.1 mm and 2.0 mm, micromachining the tubing to produce micro-machined tubing, cutting the micromachined tubing into lengths of between about 8 mm and 15 mm; and heat setting the micromachined tubing lengths to produce a flare with a flare cone angle of between 5° to 180°.

In still another aspect, the invention is a method of deploying a spacer into an opening in the human anatomy. The method includes compressing a spacer having an outer diameter of between 0.5 mm and 25 mm into a delivery device having an inner diameter of between 0.3 mm and 10 mm diameter, inserting the delivery device into the human anatomy, and deploying the spacer such that the spacer expands into the human anatomy to an outer diameter of between 0.5 mm and 25 mm.

In one embodiment, the spacer expansion may be accomplished by self-expansion, balloon expansion or a combination of self-expansion and balloon-expansion.

### BRIEF DESCRIPTION OF THE DRAWINGS

FIG. 1 is a side view of a medical device according to an embodiment of the present invention.

FIG. 2 is a side view of a medical device according to another embodiment of the present invention.

FIG. 3 is a side view of a further embodiment of the medical device according to the invention.

FIG. 4 is a side view of another embodiment of the medical device according to the invention.

FIG. 5A is a side view of yet another embodiment of the medical device according to the invention. FIG. 5B is a top view of the embodiment device of FIG. 5A.

FIG. 6A and FIG. 6B are side views of still another embodiment of the medical device according to the invention. FIG. 6C is a top view of the embodiment device of FIG. 6B.

FIG. 7 is a side view of another embodiment of the medical device according to the invention.

FIG. 8 is a side view of another embodiment of the medical device according to the invention.

FIG. 9 is a side view of a device for delivering of the medical device according to the invention.

FIG. 10 is a side view of a medical device according to another embodiment of the present invention.

### DETAILED DESCRIPTION

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

Medical devices according to embodiments of the present invention are beneficial in that, for example, their configuration provides for particularly efficient treatment of a patient's anatomy and is mechanically simple. Moreover, the simplicity of the medical devices allows for them to be manufactured in a cost effective manner.

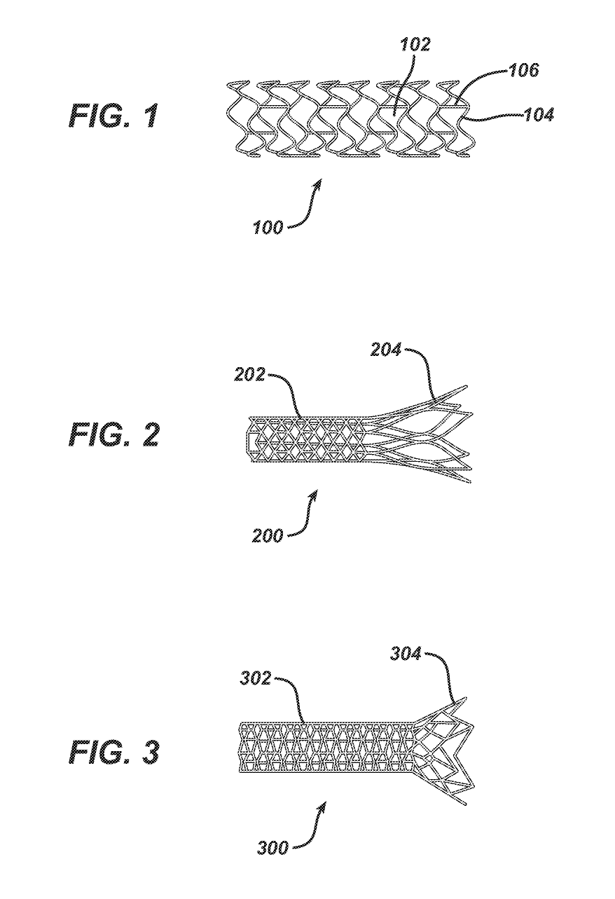

The current invention contemplates various spacer designs and scaffolds to address the above unmet clinical needs. The spacers comprise a biodegradable elastomeric polymer and my comprise any bio-absorbable polymeric system that have sufficient strength to act as a scaffolding to an opening in the human anatomy and that is bioabsorbable within about 3-4 months. In a particular embodiment, the polymer is selected from the group consisting of copolymers of caprolactone and glycolide and in a particular embodiment the polymer poliglecaprone 25 which is a 75:25 copolymer of glycolide and ε-caprolactone (PGCA). These polymers are bioabsorbable and in the case of poliglecaprone will generally absorb between within 3-4 months. Other elastomeric copolymers within the PGCA family could similary be used in this application to tailor absorption rates and ensure lateral stiffness of the material by varying the molar ratio of glycolide to ε-caprolactone or by altering the processing of the materials as is known to those of skill in the art. Higher ratios of glycolide will tend to accelerate absorption time. In general PGCA copolymers with 10%-35% ε-caprolactone are desirable to provide a material with desirable strength, elasticity, and absorption characteristics. The spacers in one embodiment are 6 mm or 7 mm in diameter and are generally sized to at least the size of the outflow tract or larger to provide for good apposition to the outflow tract of the sinus and in particular, the frontal sinus. The spacers may be small enough to be inserted into the lacrimal duct and large enough to maintain patency of the airway (between about 0.5 mm and 25 mm), which spacers may have a thickness of between about 0.1 and 2.0 mm and may be compressible into a delivery device having an inner diameter of between about 0.3 mm and 10 mm. The spacers may be between about 3 and 7 mm (OD) for use in the frontal sinus following a balloon sinuplasty procedure, may be compressible to between 2 and 3 mm in diameter and may have a thickness between about 0.25 mm and 2.0 mm. The spacers may be between about 3 mm and 15 mm for use in the frontal sinus following a FESS procedure. The spacers may be between about 1 cm and 4 cm in length, and may trimmed according to the particular procedure and anatomic peculiarities. The spacers are useful for maintaining patency of the sinus outflow tract and for preventing adhesions therein for a period of between about 3 weeks to about 3 to 4 months. The spacers may be removable after a period of time of up to about 3 weeks. The embodiments may have one, two or more longitudinal sections. The embodiment shown in FIG. 1 has one section and those of FIGS. 2-8 have two sections. These embodiments will be described in detail below.

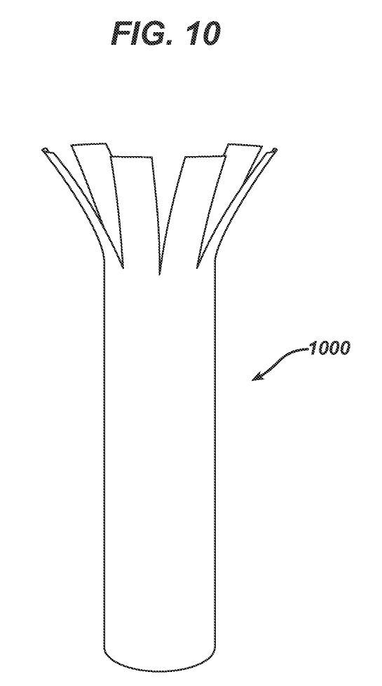

The spacer 100 shown in FIG. 1 is a 6 mm diameter×25 mm long cylindrical spacer with high radial strength. It is constructed of a copolymer of glycolide and ε-caprolactone. In particular embodiments of the invention, the copolymer may be the material known as poliglecaprone 25, a synthetic, bioabsorbable material used in the manufacture of Monocryl™ (poliglecaprone 25) suture. Monocryl suture is composed of poliglecaprone 25, which is a copolymer of glycolide (25%) and epsilon-caprolactone (75%) and comes both dyed (violet) and undyed (clear), and has a monofilament structure. The spacer 100 can be compressed to a small size (2 mm) for adequate loading into a deployment device such as a guide catheter as described in US Patent Publication No. 2009/0198216 which is hereby incorporated by reference in its entirety. The spacer device 100 may be implanted in any suitable part or location of the body of a human or animal subject to perform a spacing function (e.g., to prevent tissue ingrowth, scarring, fibrosis, adhesion formation, etc.) and/or to deliver any desired therapeutic substance. For example, in ear, nose and throat applications the spacer 100 may be implanted in a natural ostium or man-made opening formed in any paranasal sinus or air cell or in any other natural, surgically modified or surgically created opening or passageway, such as the outflow tract of a frontal sinus, the inferior, superior or medial meatus, etc. The spacer may be a solid cylinder (see spacer 1000 in FIG. 10) or may be a cylinder that has cells throughout the structure of the cylinder (see spacer 100 in FIG. 1). In the spacer 100 shown in FIG. 1, cells 102 are formed from a scaffolding of radial members 104 and longitudinal members 106. The spacer may have a proximal flare, a distal flare or both proximal and distal flares. The flare may be a shape including but not limited to conical, parabolic or tapered and continuous or it may be divided into arms or strips. When placed in the frontal sinus, the distal flare may fill or partially fill the sinus and the proximal flare may serve to inhibit the migration of the spacer into the sinus or to inhibit swallowing of the spacer. One or more spacers may be delivered according to the invention. The spacer may be positioned completely in the sinus, completely in the outflow tract or may be positioned both in the sinus and the outflow tract. The spacer will maintain apposition with the sinus or the outflow tract—as inflammation decreases it will self-expand. In post-operative procedures, it may prevent obstruction of the sinus and/or the outflow tract that would result from, for example, adhesions or scarring.

The spacer 200 shown in FIG. 2 is a 6 mm diameter×20 mm long spacer with a high radial strength cylindrical proximal section 202 and a flared distal section 204. The flared section is heat set via an annealing process at a 60° cone angle. Flared distal sections of the invention may have an angle of between about 5° and 180°. Annealing or heat setting the spacer can be achieved by heating the spacer to some temperature sufficiently above the polymer glass transition temperature (Tg) and below the melting temperature (Tm) for a sufficient time (generally 2-10 hours). The trumpet shaped flare section provides the ability of the spacer 200 to conform to the ostia of the sinus as well as to conform to the sinus anatomy. The straight tubular section provides scaffolding to the outflow tract. The spacer provides radial force to conform the device to the anatomy and help maintain patency of openings in the anatomy. In addition to conforming to the ostial region of the sinus, the trumpet shape helps conform to the sinus outflow tract, keeping the outflow tract open and optionally providing drug release to address inflammation.

The spacer 300 shown in FIG. 3 is a 6 mm diameter×30 mm long spacer with a high radial strength cylindrical proximal section 302 and a flared distal section 304 showing a different flare geometry.

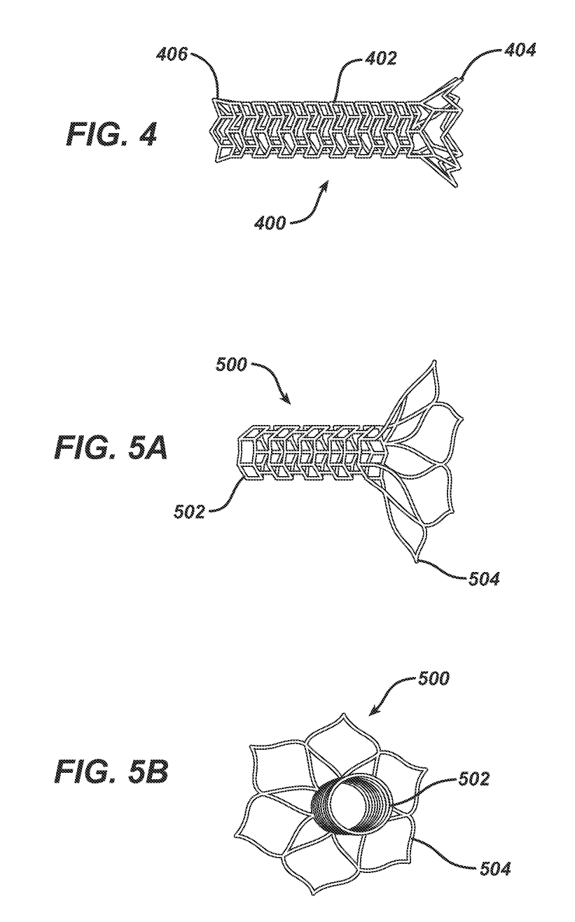

The spacer 400 shown in FIG. 4 is a 6 mm diameter×30 mm long spacer with a high radial strength cylindrical middle section 402 and flared distal and proximal sections 404 and 406 respectively. The disal and proximal flare sections provide for improved retention of the spacer 400 in the sinus anatomy at both the exit and entrance of the outflow tract.

The spacer 500 shown in FIG. 5 is a 6 mm diameter×20 mm long spacer with a high radial strength cylindrical proximal section 502 and a flared distal section 504. The flare is set at a 140° cone angle.

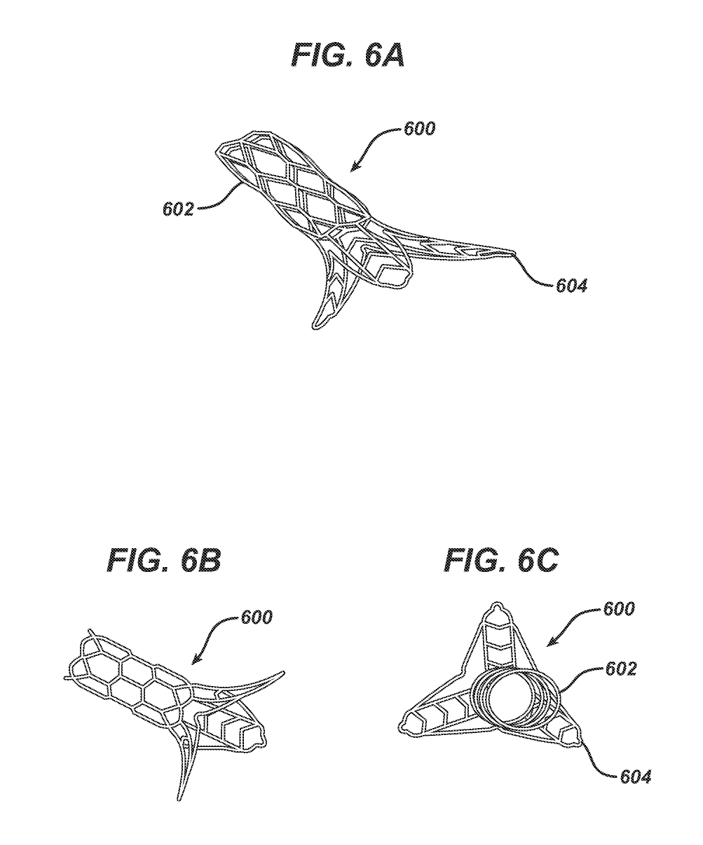

The spacer 600 shown in FIG. 6A is a 6 mm diameter×20 mm long spacer and in 6B and 6C is a 7 mm diameter×20 mm long spacer with high radial strength cylindrical proximal sections 602 and a flared distal sections 604. The flare is set at a 140° cone angle. The construction of the spacer scaffolding, with longitudinal members that are in line with the flow of mucus (there are a greater number of longitudinal members than axial members) enables mucus flow and clearing of the drainage pathway following spacer deployment.

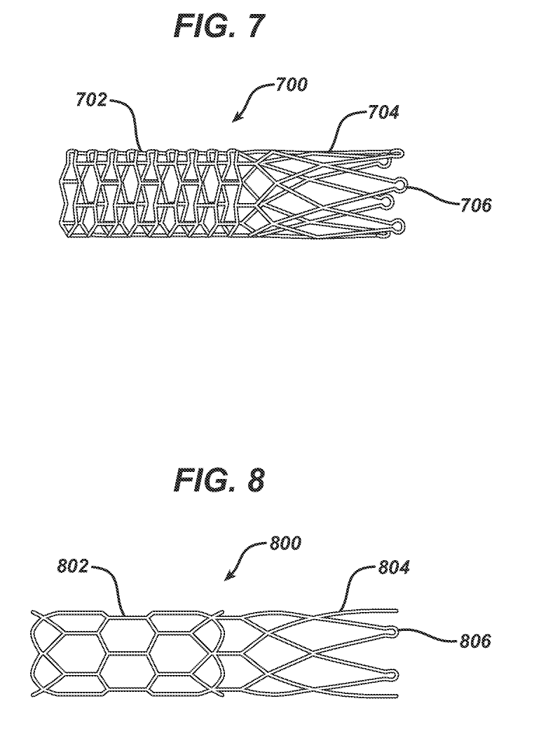

The spacer 700 shown in FIG. 7 is 6 mm diameter×20 mm long spacer with a high radial strength cylindrical proximal section 702 and a deformable tulip flare distal section 704. The deformable tulip flare coupled with a soft eyelet terminus 706 provides for good sinus anatomy apposition. In this embodiment, a violet dye is included in the polymer for greater visibility during endoscopy both during and following deployment of the spacer in the sinus anatomy.

The spacer 800 shown in FIG. 8 is a 7 mm diameter×20 mm long spacer with a high radial strength cylindrical proximal section 802 and a deformable tulip flare distal section 804. The deformable tulip flare coupled with a soft eyelet terminus 806 and the greater 7 mm diameter provides for good sinus anatomy apposition. The construction of the spacer scaffolding, with longitudinal members that are in line with the flow of mucus (there are a greater number of longitudinal members than axial members) enables mucus flow and clearing of the drainage pathway following spacer deployment. In this embodiment, a violet dye is included in the polymer for greater visibility during endoscopy both during and following deployment of the spacer in the sinus anatomy.

In addition to its use for maintaining the patency of the site of deployment, the spacers of the current invention can also be used to deliver diagnostic or therapeutic substances into the sinuses or other areas in the paranasal space or spaces of the ear, nose or throat. The substances may be incorporated into the scaffolding of the spacers of the invention or may be deposited onto the surfaces of the spacers. The substances include diagnostic or therapeutic substances, which substances are include any feasible drugs, prodrugs, proteins, genes or gene therapy preparations, cells including stem cells, diagnostic agents, contrast or imaging agents, biologicals, etc. Such substances may be in bound or free form, liquid or solid, colloid or other suspension, solution or may be in the form of a gas or other fluid or non-fluid. For example, the substance delivered may comprise pharmaceutically acceptable salt or dosage form of an antimicrobial agent (e.g., antibiotic, antiviral, antiparasitic, antifungal, etc.), an anesthetic agent with or without a vasoconstriction agent (e.g. lidocaine, xylocalne or tetracaine, each with or without epinephrine, etc.), a corticosteroid or other anti-inflammatory (e.g., an NSAID), a decongestant (e.g., vasoconstrictor), a mucous thinning agent (e.g., an expectorant or mucolytic), an agent that prevents or modifies an allergic response (e.g., an antihistamine, cytokine inhibitor, leucotriene inhibitor, IgE inhibitor, immunomodulator), a surfactant, hemostatic agents to stop bleeding, antiproliferative agents, an allergen or another substance that causes secretion of mucous by tissues, hemostatic agents to stop bleeding, antiproliferative agents, cytotoxic agents (e.g. alcohol), and the like.

Some nonlimiting examples of antimicrobial agents that may be used in this invention include acyclovir, amantadine, aminoglycosides (e.g., amikacin, gentamicin and tobramycin), amoxicillin, amoxicillinlclavulanate, amphotericin B, ampicillin, ampicillinlsulbactam, atovaquone, azithromycin, cefazolin, cefepime, cefotaxime, cefotetan, cefpodoxime, ceflazidime, ceflizoxime, ceftriaxone, cefuroxime, cefuroxime axetil, cephalexin, chloramphenicol, clotrimazole, ciprofloxacin, clarithromycin, clindamycin, dapsone, dicloxacillin, doxycycline, erythromycin, fluconazole, foscamet, ganciclovir, atifloxacin, imipenemlcilastatin, isoniazid, itraconazole, ketoconazole, metronidazole, nafcillin, nafcillin, nystatin, penicillin, penicillin G, pentamidine, piperacillinitazobactam, rifampin, quinupristindalfopristin, ticarcillinlclavulanate, trimethoprimlsulfamethoxazole, valacyclovir, vancomycin, mafenide, silver sulfadiazine, mupirocin (e.g., Bactroban, Glaxo SmithKline, Research Triangle Park, N.C.), nystatin, triamcinolonelnystatin, clotrimazolelbetamethasone, clotrimazole, ketoconazole, butoconazole, miconazole, tioconazole, detergent-like chemicals that disrupt or disable microbes (e.g., nonoxynol-9, octoxynol-9, benzalkonium chloride, menfegol, and N-docasanol); chemicals that block microbial attachment to target cells and/or inhibits entry of infectious pathogens (e.g., sulphated and sulphonated polymers such as PC-515 (carrageenan), Pro-2000, and Dextrin 2 Sulphate); antiretroviral agents (e.g., PMPA gel) that prevent retroviruses from replicating in the cells; genetically engineered or naturally occurring antibodies that combat pathogens such as anti-viral antibodies genetically engineered from plants known as “plantibodies;” agents which change the condition of the tissue to make it hostile to the pathogen (such as substances which alter mucosal pH (e.g., Buffer Gel and Acid form); non-pathogenic or “friendly” microbes that cause the production of hydrogen peroxide or other substances that kill or inhibit the growth of pathogenic microbes (e.g., lactobacillus); antimicrobial proteins or peptides such as those described in U.S. Pat. No. 6,716,813 (Lin et al.) which is expressly incorporated herein by reference or antimicrobial metals (e.g., colloidal silver).

Additionally or alternatively, in some applications where it is desired to treat or prevent inflammation the substances delivered in this invention may include various steroids or other anti-inflammatory agents (e.g., nonsteroidal anti-inflammatory agents or NSAIDS), analgesic agents or antipyretic agents. For example, corticosteroids that have previously administered by intranasal 10 administration may be used, such as beclomethasone (Vancenase® or Beconase), flunisolide (Nasalid®), fluticasone proprionate (Flonase®), triamcinolone acetonide (Nasacort®), budesonide (Rhinocort Aqua®), loterednol etabonate (Locort) and mometasone (Nasonex®). Other salt forms of the aforementioned corticosteroids may also be used. Also, other non-limiting examples of steroids that may be useable in the present invention include but are not limited to aclometasone, desonide, hydrocortisone, betamethasone, clocortolone, desoximetasone, fluocinolone, flurandrenolide, mometasone, prednicarbate; amcinonide, desoximetasone, diflorasone, fluocinolone, fluocinonide, halcinonide, clobetasol, augmented betamethasone, diflorasone, halobetasol, prednisone, dexarnethasone and methylprednisolone. Other anti-inflammatory, analgesic or antipyretic agents that may be used include the nonselective COX inhibitors (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone) and Selective COX-2 Inhibitors (e.g., diaryl-substituted furanones such as rofecoxib; diaryl-substituted pyrazoles such as celecoxib; indole acetic acids such as etodolac and sulfonanilides such as mesulide).

Additionally or alternatively, in some applications, such as those where it is desired to treat or prevent an allergic or immune response and/or cellular proliferation, the substances delivered in this invention may include a) various cytokine inhibitors such as humanized anti-cytokine antibodies, anti-cytokine receptor antibodies, recombinant (new cell resulting from genetic recombination) antagonists, or soluble receptors; b) various leucotriene modifiers such as zafirlukast, montelukast and zileuton; c) immunoglobulin E (IgE) inhibitors such as Omalizumab (an anti-IgE monoclonal antibody formerly called rhu Mab-E25) and secretory leukocyte protease inhibitor) and d) SYK Kinase inhibitoers such as an agent designated as “R-112” manufactured by Rigel Pharmaceuticals, Inc, South San Francisco, Calif.

Additionally or alternatively, in some applications, such as those where it is desired to shrink mucosal tissue, cause decongestion, or effect hemostasis, the substances delivered in this invention may include various vasoconstrictors for decongestant and or hemostatic purposes including but not limited to pseudoephedrine, xylometazoline, oxymetazoline, phenylephrine, epinephrine, etc.

Additionally or alternatively, in some applications, such as those where it is desired to facilitate the flow of mucous, the substances delivered in this invention may include various mucolytics or other agents that modify the viscosity or consistency of mucous or mucoid secretions, including but not limited to acetylcysteine. In one particular embodiment, the substance delivered by this invention comprises a combination of an anti-inflammatory agent (e.g. a steroid or an NSAID) and a mucolytic agent.

Additionally or alternatively, in some applications such as those where it is desired to prevent or deter histamine release, the substances delivered in this invention may include various mast cell stabilizers or drugs which prevent the release of histamine such as crornolyn (e.g., Nasal Chroma) and nedocromil.

Additionally or alternatively, in some applications such as those where it is desired to prevent or inhibit the effect of histamine, the substances delivered in this invention may include various antihistamines such as azelastine (e.g., Astylin) diphenhydramine, loratidine, etc.

Additionally or alternatively, in some embodiments such as those where it is desired to dissolve, degrade, cut, break or remodel bone or cartilage, the substances delivered in this invention may include substances that weaken or modify bone and/or cartilage to facilitate other procedures of this invention wherein bone or cartilage is remodeled, reshaped, broken or removed. One example of such an agent would be a calcium chelator such as EDTA that could be injected or delivered in a substance delivery implant next to a region of bone that is to be remodeled or modified. Another example would be a preparation consisting of or containing bone degrading cells such as osteoclasts. Other examples would include various enzymes of material that may soften or break down components of bone or cartilage such as collagenase (CGN), trypsin, trypsinlLEDTA, hyaluronidase, and tosyllysylchloromethane (TLCM).

Additionally or alternatively, in some applications, the substances delivered in this invention may include other classes of substances that are used to treat rhinitis, nasal polyps, nasal inflammation, and other disorders of the ear, nose and throat including but not limited to anti-cholinergic agents that tend to dry up nasal secretions such as ipratropium (Atrovent Nasal®), as well as other agents not listed here.

Additionally or alternatively, in some applications such as those where it is desired to draw fluid from polyps or edematous tissue, the substances delivered in this invention may include locally or topically acting diuretics such as furosemide and/or hyperosmolar agents such as sodium chloride gel or other salt preparations that draw water from tissue or substances that directly or indirectly change the osmolar content of the mucous to cause more water to exit the tissue to shrink the polyps directly at their site.

Additionally or alternatively, in some applications such as those wherein it is desired to treat a tumor or cancerous lesion, the substances delivered in this invention may include antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as; alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other antitumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase inhibitors such as aminoglutethamide and formestane, trriazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-I, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU11248, BMS-275291, COL-3, EMD 121974, 1MC-IC11, 1M862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000) which is expressly incorporated herein by reference, biological response modifiers (e.g., interferon, bacillus calmetteguerin (BCG), monoclonal antibodies, interluken 2, granulocyte colony stimulating factor (GCSF), etc.), PGDF receptor antagonists, herceptin, asparaginase, busulphan, carboplatin, cisplatin, carmustine, cchlorambucil, cytarabine, dacarbazine, etoposide, flucarbazine, fluorouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol, taxotere, analogslcongeners and derivatives of such compounds as well as other antitumor agents not listed here.

Additionally or alternatively, in some applications such as those where it is desired to grow new cells or to modify existing cells, the substances delivered in this invention may include cells (mucosal cells, fibroblasts, stem cells or genetically engineered cells) as well as genes and gene delivery vehicles like plasmids, adenoviral vectors or naked DNA, mRNA, etc. injected with genes that code for anti-inflammatory substances, etc., and, as mentioned above, osteoclasts that modify or soften bone when so desired, cells that participate in or effect mucogenesis or ciliagenesis, etc.

Spacers of these embodiments are conducive to manufacturing by a variety of methods. One method is by laser micromachining polymer tubing. The polymer tubing can be produced, for example, by melt extrusion, injection molding, and solvent casting. Other manufacturing methods may consist of injection or compression molding the design, and then wrapping the spacer around a mandrel, and welding (i.e. using ultrasound, laser, or heat) the lateral ends to form a cylindrical or tapered shape. Following manufacturing of the spacer geometry the spacer can be placed on a tapered mandrel and annealed at the appropriate temperature/time to heat set the flare geometry at either end of the spacer.

Pharmaceutical agents can be applied as is known the current spacer manufacturing art. One typical method is to solubulize the drug in a solvent solution with a polymer and either dip or spray coat the solution onto the spacer as a coating. Other means are provided to apply the drug directly as a coating without a polymer. Other means are to include the pharmaceutical agent into the melt or solution of the polymer used to construct the spacer prior to its manufacture into a cylindrical shape or scaffolding.

The current invention contemplates various spacer designs and scaffolds to address the above unmet clinical needs. The spacers comprise a biodegradable elastomeric polymer. In a particular embodiment, the polymer is selected from the group consisting of copolymers of caprolactone and glycolide and in a particular embodiment the polymer poliglecaprone 25 is a 75:25 copolymer of glycolide and epsilon-caprolactone (PGCA). These polymers are bioabsorbable and in the case of poliglecaprone will generally absorb between within 3-4 months. Other elastomeric copolymers within the PGCA family could similary be used in this application to tailor absorption rates by varying the molar ratio of glycolide to epsilon-caprolactone. Higher ratios of glycolide will tend to accelerate absorption time. In general, PGCA copolymers with 10%-35% epsilon-caprolactone are desirable to provide a material with desirable strength, elasticity, and absorption characteristics. The spacers in one embodiment are 6 mm or 7 mm in diameter and are generally sized to at least the size of the outflow tract or larger to provide for good apposition to the outflow tract of the sinus and in particular, the frontal sinus. The embodiments may have one, two or more longitudinal sections.

The polymer tubing that is provided may be prepared by conventional methods such as extrusion, injection molding, and solvent casting. The desired polymer tubing diameter and wall thickness are dependent on the final diameter of the spacer. In one embodiment of the present invention, the polymeric intraluminal spacers are first prepared by providing a cylindrical polymer tube having a diameter of between 6 mm and 7 mm and approximately 0.25 mm in thickness. The PCGA copolymer, such as poliglecaprone 25 or poly(glycolide/caprolactone (75/25) can be melt extruded at a temperature of 220 C using a conventional screw extruder. The polymer tubing is then processed to provide a spacer with the desired spacer configuration by cutting the tubing to the desired length and then machining to obtain the desired geometric configuration. Machining of the spacer may be accomplished by conventional methods such as laser micromachining using a low energy laser. After the spacer is prepared various flared end shapes can be introduced as follows. A metal mandrel is machined with the desired flare geometry such as a cone-shape. The cone angle can range from 5 degrees to 180 degrees or more depending on the specific strut geometry and material used. The spacer is mounted snugly on the mandrel and then annealed or heated (through any known means) for a period of time suitable for the copolymer to crystallize to a desired degree necessary to hold the geometrical shape. Annealing serves also to promote product stability. The heating process used to shape set the spacer flared end will depend on the specific PCGA copolymer material used, but may require a temperature of 75 C-110 C for a period of 3-10 hrs to heat set the flare geometry at either end of the stent. For example polymer tubing made of 75/25 (mol/mol) glycolide and epsilon-caprolactone (PGCA) was extruded with an outer diameter (OD) of 6.0 mm and an inner diameter (ID) of 5.50 mm Spacers having geometries shown in the figures such as FIG. 3 (flared stent) were laser cut from the tubing using a low energy laser. The spacers were mounted on metal mandrels with a tapered conical angle of either 60 degrees or 140 degrees and placed in a nitrogen purged oven set at 76 C where the stents were heated for 17.5 hours. After heating the spacers were cooled to room temperature in a vacuum oven and removed from the mandrels. The flared end geometry of the stent was maintained.

A second manufacturing method consists of compression molding the spacer design in a flat mold and subsequently post-processing the molded piece into a cylindrical shape by wrapping the spacer around a mandrel, and bonding the longitudinal seam through known means such as adhesives or welding (ultrasonic, laser, heat, or other known means) to form a cylindrical or tapered shape with a longitudinal axis. In one embodiment, the mandrel can have a tapered or conical-shaped end which will produce the flared end of the spacer. The spacer is then annealed

The spacers of the disclosed invention may be provided with therapeutic substances, including but not limited to triamcinolone acetonide. The therapeutic substance may be incorporated into the spacer in different ways. For example, the therapeutic substances may be coated onto the spacer, after the spacer has been formed, wherein the coating is comprised of polymeric materials into which therapeutic substances are incorporated. There are several ways to coat the spacers. Some of the commonly used methods include spray coating; dip coating; electrostatic coating; fluidized bed coating; and supercritical fluid coatings. Alternately, the therapeutic substances may be incorporated into the polymeric materials comprising the spacer. The therapeutic substance may be blended into the polymer melt just prior to extrusion into a tube. The therapeutic substance can be housed in reservoirs or wells in the spacer configuration. These various techniques of incorporating therapeutic substances into, or onto, the spacer may also be combined to optimize performance of the spacer, and to help control the release of the therapeutic substances from the spacer. Coatings may be applied to the spacer in a variety of processes known in the art such as, for example, chemical vapor deposition (CVD), physical vapor deposition (PVD), electroplating, high-vacuum deposition process, microfusion, spray coating, dip coating, electrostatic coating, or other surface coating or modification techniques.

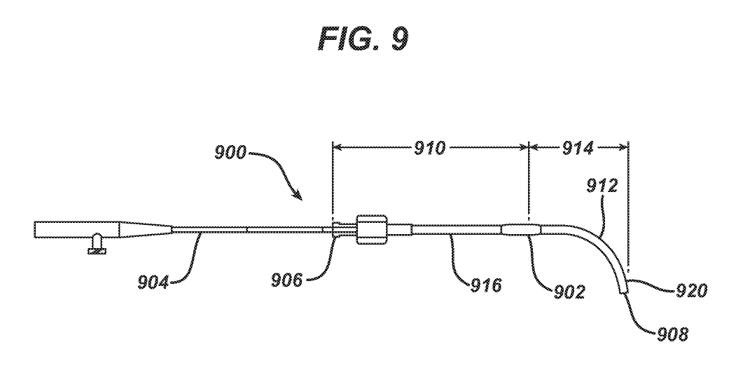

FIG. 9 shows one embodiment of a delivery system 900 for a bioabsorbable spacer according to the invention. The delivery system 900 includes a delivery guide 902 and a spacer deployment device 904, and optionally a guide element. The delivery guide 902 has a proximal end 906 and a distal end 908, each having inner diameters of 0.10 inches. The outer diameter of the distal end 908 is 0.135 inches and of the proximal end 906 is 0.154 inches. The total axial length 910 of the device is 14 cm, the distal shaft 912 is a nylon shaft and is clear and has an axial length 914 of 5 to 6 cm. The proximal shaft portion 916 has is a stainless steel hypotube with a PTFE liner and a nylon outer shaft. The particular embodiment shown in FIG. 9 is particularly useful to deliver a spacer to the frontal sinus. Different curvatures are useable to access the different sinuses. For example, a 0 to 30 degree delivery guide is typically used to access the sphenoid or the ethmoid sinus, a 50 to 90 degree delivery guide is typically used to access the frontal sinus, and a 70 to 110 degree guide is typically used to access the maxillary sinus.

In one embodiment of the delivery system 900, the spacer deployment device 904 is a spacer pusher. For delivery of the spacer (i.e. spacer 100 shown above) using a spacer pusher, the spacer 100 is preloaded into the delivery guide 902 and the spacer pusher is inserted into the delivery guide 902. The spacer 100 is positioned at the delivery guide tip 920 (an atraumatic tip which may include a colored marker for endoscopic visualization) using the spacer pusher. Under endoscopy the guide tip 920 is positioned in the target anatomy (in this case the frontal sinus) and the spacer pusher is advanced to position the spacer at the target anatomy.

In one embodiment of the delivery system 900, the spacer deployment device 904 is a balloon catheter such as those manufactured by Acclarent, Inc for use in balloon sinuplasty procedures. For delivery of the spacer (i.e. spacer 100 shown above) using a balloon catheter, the spacer 100 is preloaded into the delivery guide 902 and the balloon catheter is inserted into the delivery guide 902. The spacer 100 is positioned at the delivery guide tip 920 (an atraumatic tip which may include a colored marker for endoscopic visualization) by advancing the balloon catheter within the delivery guide 902. Under endoscopy the guide tip 920 is positioned in the target anatomy (in this case the frontal sinus) either before or after a balloon dilation procedure or a FESS procedure (functional endoscopic sinus surgery procedure). A guide element (a guide wire or an illuminating guide wire) which may be preloaded into delivery system 900 is then advanced through the catheter lumen and into the target anatomy. The balloon catheter is advanced to position the spacer in the target anatomy and the balloon is inflated to fully deploy the spacer (See US Patent Publication No. 2009/0198216 referenced above). The spacer may be self-expandable, balloon expandable, or a combination of the two. The spacer may be deployed and redeployed or repositioned within the sinus anatomy without damage to the spacer.

The below examples serve to further describe the invention but not to limit it in any way.

### Example #1

### Lateral Compression Testing

Lateral compression testing was performed various spacer designs manufactured from three different extruded tubings (6.0 mm (violet dyed and undyed) and 7.0 mm violet dyed tubing) in several designs shown in the figures. Prototype spacers tested were not sterilized prior to testing. For purposes of testing the mechanical properties of the spacer region, the flared end of the spacer to be tested was first removed by sectioning with a razor. The spacer region was mounted between two flat platens of an Instron tensile test machine (Model 5565, Bluehill software) equipped with a 10N load cell, so the spacer could be compressed perpendicular to the longitudinal axis of the stent. A lateral compressive force was applied to the spacer at a rate of 5 mm/min until the spacer was compressed 2 mm. The lateral stiffness in (kPa) was automatically recorded as a Young's modulus by the test program for the initial linear region of the force/deflection curve and were subsequently converted to Stiffness (N/mm). Tests were conducted in air at room temperature. After testing at day zero, the spacers placed in phosphate buffered saline (Sigma) and placed on a low cycle stir plate in an environmental chamber set at 37 C. At day 4 and day 6 the samples were removed and tested in repeat fashion. The results are shown in Table 1.

TABLE 1

Stiffness (N/mm)

| | Tubing | Day 0 | Day 4 | Day 6 |
|---|---|---|---|---|
| FIG. 3 | 6.0 mm, undyed | 2.48 | 2.30 | 2.51 |
| FIG. 2 | 6.0 mm, undyed | 2.28 | 2.03 | 2.25 |
| FIG. 2 | 6.0 mm, dyed | 2.22 | 2.10 | 2.22 |
| FIG. 6A | 6.0 mm, undyed | 0.57 | 0.52 | 0.58 |
| FIG. 6B | 7.0 mm, dyed | 0.77 | 0.75 | 0.85 |

In general the spacers exhibited a 5%-10% drop in lateral stiffness by Day 4, but by Day 6 this drop was negligible. This demonstrates that spacers made from the disclosed methods and designs largely retained lateral stiffness through the first 6 days under simulated conditions which is crucial for the spacers to afford mechanical support during the healing phase.

### Example #2

### 14 Day Absorption

An unsterilized violet-dyed spacer shown in FIG. 2 was placed in phosphate buffered saline (Sigma) and placed on a low cycle stir plate in an environmental chamber set at 37C. The spacer was removed from the chamber periodically and visually checked after swirling the sample in the saline. After 14 days the spacers physically remained intact indicating that a degree of mechanical integrity remained.

The invention has been described with reference to certain examples or embodiments of the invention, but various additions, deletions, alterations and modifications may be made to those examples and embodiments without departing from the intended spirit and scope of the invention. For example, any element or attribute of one embodiment or example may be incorporated into or used with another embodiment or example, unless otherwise specified or if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

What is claimed is:

1. A spacer for maintaining the patency of an opening in the sinus anatomy said spacer comprising an absorbable co-polymer of epsilon-caprolactone and glycolide said co-polymer having between 10% and 35% by weight of epsilon-caprolactone.

2. The spacer of claim 1 wherein the spacer is in the shape of a cylinder.

3. The spacer of claim 2 wherein the cylinder has a proximal end and a distal end and wherein the distal end comprises a flare having an angle of between 5 degrees and 180 degrees.

4. The spacer of claim 3 wherein the proximal end further comprises a flare.

5. The spacer of claim 2 wherein the cylinder comprises a scaffolding.

6. The spacer of claim 5 wherein the scaffolding comprises members selected from the group consisting of longitudinal members, axial members and a combination of longitudinal and axial members.

7. The spacer of claim 1 having an initial lateral stiffness of between about 0.2 and 5 N/mm.

8. The spacer of claim 7 having an initial lateral stiffness of between about 0.5 and 2.5 N/mm.

9. The spacer of claim 1 further comprising a therapeutic or diagnostic substance.

10. The spacer of claim 9 wherein the therapeutic substance is triamcinolone acetonide.

11. A method of making a spacer for maintaining the patency of an opening in the sinus anatomy, the method comprising:
a. melting an extrusion of the a copolymer of epsilon-caprolactone and glycolide to make a tubing of between 6 mm and 7 mm in diameter with a thickness of between 0.5 mm an 2.0 mm;
b. micromachining the tubing to produce micronmachined tubing;
c. cutting the micromachined tubing into micromachined tubing lengths of between about 8 mm and 15 mm; and
d. heat setting the micromachined tubing lengths to produce a flare with a flare cone angle of between 5° to 180°.

12. A method of deploying a spacer into an opening in the sinus anatomy to maintain the patency of the opening, the method comprising:
a. compressing a spacer having an outer diameter of between 6 mm and 7 mm into a delivery device having an inner diameter of between 2 mm and 3 mm diameter;
b. inserting the delivery device into the sinus anatomy; and
c. deploying the spacer such that the spacer expands into the sinus anatomy to an outer diameter of between 6 mm and 7 mm.

13. The method of claim 12 wherein the spacer expands by a method selected from the group consisting of self-expansion, balloon expansion or a combination of self-expansion and balloon-expansion

14. A method for delivering triamcinolone acetonide to the frontal sinus anatomy, the method comprising;
a. compressing a spacer having an outer diameter of between 6 mm and 7 mm and comprising triamcinolone acetonide into a delivery device having an inner diameter of between 2 mm and 3 mm diameter;
b. inserting the delivery device into the frontal sinus anatomy; and
c. deploying the spacer such that the spacer expands into the frontal sinus anatomy to an outer diameter of between 6 mm and 7 mm and delivers the triamcinolone acetonide into the frontal sinus anatomy.

15. The method of claim 14 wherein the spacer expands by a method selected from the group consisting of self-expansion, balloon expansion or a combination of self-expansion and balloon-expansion.

16. A spacer for maintaining an opening in the anatomy of a human patient, said opening being accessible from the ear, nose or throat of the human patient, the spacer comprising an absorbable co-polymer of epsilon-caprolactone and glycolide said co-polymer having between 10% and 35% by weight of epsilon-caprolactone.

17. The spacer of claim 16 wherein the spacer is in the shape of a cylinder.

18. The spacer of claim 17 wherein the cylinder has a proximal end and a distal end and wherein the distal end comprises a flare having an angle of between 5 degrees and 180 degrees.

19. The spacer of claim 18 wherein the proximal end further comprises a flare.

20. The spacer of claim 17 wherein the cylinder comprises a scaffolding.

21. The spacer of claim 20 wherein the scaffolding comprises members selected from the group consisting of longitudinal members, axial members and a combination of longitudinal and axial members.

22. The spacer of claim 16 having an initial lateral stiffness of between about 0.2 and 5 N/mm.

23. The spacer of claim 22 having an initial lateral stiffness of between about 0.5 and 2.5 N/mm.

24. The spacer of claim 23 further comprising a therapeutic agent

25. A method of making a spacer for maintaining an opening in the human anatomy, the method comprising:
a. melting an extrusion of the a copolymer of epsilon-caprolactone and glycolide to make a tubing of between 0.5 mm and 25 mm in diameter with a thickness of between 0.1 mm and 2.0 mm;
b. micromachining the tubing to produce micronmachined tubing;
c. cutting the micromachined tubing into micromachined tubing lengths of between about 8 mm and 15 mm; and
d. heat setting the micromachined tubing lengths to produce a flare with a flare cone angle of between 5° to 180°.

26. A method of deploying a spacer into an opening in the human patient to maintain the opening, the opening being accessible from the ear, nose or throat of the human patient, the method comprising:
a. compressing a spacer having an outer diameter of between 0.5 mm and 25 mm into a delivery device having an inner diameter of between 0.3 mm and 10 mm diameter;
b. inserting the delivery device into the ear, nose or throat of the human patient; and
c. deploying the spacer such that the spacer expands into the opening to an outer diameter of between 0.5 mm and 25 mm.

27. The method of claim 26 wherein the spacer expands by a method selected from the group consisting of self-expansion, balloon expansion or a combination of self-expansion and balloon-expansion
